(19) United States
(12) Patent Application Publication
(10) Pub. No.: US 2011/0270321 A1
(43) Pub. Date: Nov. 3, 2011

(54) Engaging Member With a Cavity-Base for Engaging a Connecting Element to a Bone Anchor

(75) Inventors: Julien J. Prevost, Memphis, TN (US); Robert M. Loke, Memphis, TN (US); Matthew Van Nortwick, Memphis, TN (US); Douglas N. Baker, Collierville, TN (US); Douglas D. Kave, Byhalia, MS (US)

(73) Assignee: WARSAW ORTHOPEDIC, INC., Warsaw, IN (US)

(21) Appl. No.: 12/771,106

(22) Filed: Apr. 30, 2010

(51) Int. Cl. A61B 17/86

(52) U.S. Cl. 606/305

(57) ABSTRACT

Devices and methods include an anchor assembly engageable to a vertebra and a connecting element positionable through a receiver of the anchor assembly. The assembly includes an engaging member for engaging the connecting element in the receiver. The engaging member includes a base with a cavity that receives a deformed portion the connecting element when the engaging member is securely engaged to the connecting element.

### BACKGROUND

The present invention concerns engaging members and bone anchors, particularly useful for engaging a connecting element to one or more vertebrae. In a particular embodiment, the invention contemplates a bone anchor assembly with an engaging member having a cavity-base for engaging the connecting element to the bone anchor.

Several techniques and systems have been developed for correcting and stabilizing the spine and for facilitating fusion at various levels of the spine. In one type of system, a connecting element such as an elongated rod is disposed longitudinally along the length of the spine or several vertebrae of the spinal column. The rod may be bent to correspond to the normal or desired curvature of the spine in the particular region being instrumented. For example, the rod can be bent or angled to form a normal kyphotic curvature for the thoracic region of the spine, or a lordotic curvature for the lumbar region. In accordance with such a system, the rod is engaged to various vertebrae along the length of the spinal column by way of a number of bone anchors. A variety of bone anchors can be provided which are configured to engage specific portions of the vertebra. For instance, one such bone anchor is a hook that is configured to engage the lamina of the vertebra. Another type of bone anchor is a spinal screw which can be threaded into one or more aspects of the vertebral bone, such as the pedicle.

In one typical procedure utilizing a bendable, angled or linear rod, one or more of the rods is situated on one or both of the opposite sides of the spine or spinous processes. A plurality of bone screws are threadingly engaged to several vertebral bodies, such as to the pedicles of these vertebrae. One or more of the bone screws are maneuvered to manipulate the position or orientation of the vertebral body or bodies to which the bone screw is engaged. The rod(s) are connected or affixed to the plurality of bone screws to apply and maintain corrective and stabilizing forces to the spine.

The bone anchors in spinal procedures can have receivers with channels for the connecting element that, in some bone anchors, open upward, i.e. directly away from the bone to which the anchor is attached. Other bone anchors utilize channels that open along the medial or lateral side of the anchor to receive the connecting element. It is desirable in some procedures to utilize an engaging member to secure the connecting element to the bone anchor in the channel. However, the force applied to engage the engaging member to the bone anchor and the connecting element can introduce undesired stress into the construct. Additional improvements in the engagement of connecting elements to bone anchors are still needed.

### SUMMARY

The present invention generally relates to devices and methods that securely engage an elongated connecting element with a bone anchor. The bone anchor includes a bone engaging portion engageable to bony structure and a receiver that includes a passage or other structure for receiving the connecting element when the connecting element is positioned along the bony structure. An engaging member is engaged to the receiver and the connecting element to secure the connecting element with the bone anchor.

According to one aspect, the engaging member includes a base that is engaged to the bone anchor and a distal face at one end of the base. The distal face includes a cavity extending proximally into the base. The cavity receives a portion of the connecting element therein so that when the engaging member is securely engaged to the bone anchor with the distal face pressing against the connecting element, the engaging member deforms the connecting element into the cavity without penetrating the connecting element.

According to another aspect, a bone anchor assembly includes a bone engaging portion and a receiver extending proximally from the bone engaging member. The receiver defines a passage that receives an elongate connecting element therein. The connecting element is made from a polymeric material. The bone anchor assembly also includes an engaging member that is engaged to the receiver with a distal face of the engaging member facing the connecting element. The engaging member includes a cavity extending therein from the distal face, and the distal face defines a pressure surface extending around the cavity. Sufficient torque is applied to the engaging member so that the pressure surface of the distal face presses against the connecting element to deform the connecting element into the cavity.

According to another aspect, a spinal implant assembly includes a bone anchor member with a distal bone engaging portion engageable to a spinal column and a receiver at a proximal end of the bone engaging portion that defines a passage. An elongate connecting element is positionable along the spinal column through the passage of said receiver. The assembly also includes an engaging member engageable with the receiver. The engaging member extends along a rotation axis between a distal end and a proximal end. The distal end of the engaging member includes a pressure surface extending around the rotation axis. The pressure surface also extends around a cavity. The cavity extends proximally from the pressure surface into the engaging member to an end wall in the engaging member that is recessed from the pressure surface. The cavity receives a portion of the connecting element that deforms into the cavity when the pressure surface is compressingly engaged to the connecting element in the receiver.

Related features, aspects, embodiments, objects and advantages of the present invention will be apparent from the following description and the drawings.

### BRIEF DESCRIPTION OF THE DRAWINGS

FIG. 1 is a section view of a portion of an anchor assembly with a connecting element positioned therein.

FIG. 2 is a sectional view along the rotational axis of a distal part of the engaging member of FIG. 1 with the engaging member securely engaged to the connecting element.

FIG. 3 is a sectional view showing the base portion of the engaging member engaged to a connecting element in another embodiment anchor assembly.

FIG. 4 is a side elevation view of the engaging member of FIG. 1.

FIG. 5 is an elevation view of the proximal end of the engaging member of FIG. 4.

FIG. 6 is an elevation view of the distal end of the engaging member of FIG. 4.

FIG. 7 is a section view of the engaging member along line 7-7 of FIG. 4.

FIG. 8 is an enlarged view of the distal portion of the section view of the engaging member in FIG. 7.

FIG. 9 is a perspective view of the engaging member of FIG. 1.

FIG. 10 is a perspective view of another embodiment engaging member.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

Devices and methods for securing a connecting element in an anchor assembly are provided for spinal and other bone stabilization surgical procedures. The anchor assembly includes a bone anchor and an engaging member engaged to the bone anchor. The engaging member includes a distal face that has a cavity and a pressure surface around the cavity. The pressure surface contacts and deforms the outer surface of the connecting element into the cavity to securely engage the connecting element to the bone anchor. The engaging member does not bite into or penetrate the connecting element, avoiding the creation of locations in the connecting element that could develop cracks or fractures when the connecting element is loaded, thus preserving the structural integrity of the connecting element. Since the deformation of the connecting element is maintained within the bone anchor assembly, the performance and integrity of the deformed connecting element in resisting spinal loads is not diminished. Furthermore, the amount of torque required to be applied to the engaging member to secure the connecting element with the desired axial grip in the bone anchor is reduced since the cavity of the engaging member captures a portion of the connecting element that is deformed into the cavity. Thus, axial movement of the connecting element relative to the bone anchor and engaging member is not only resisted by the frictional engagement between the pressure surface of the engaging member and the connecting element, but also by the resistance to axial shear forces developed by the portion of the connecting element that is received in the cavity.

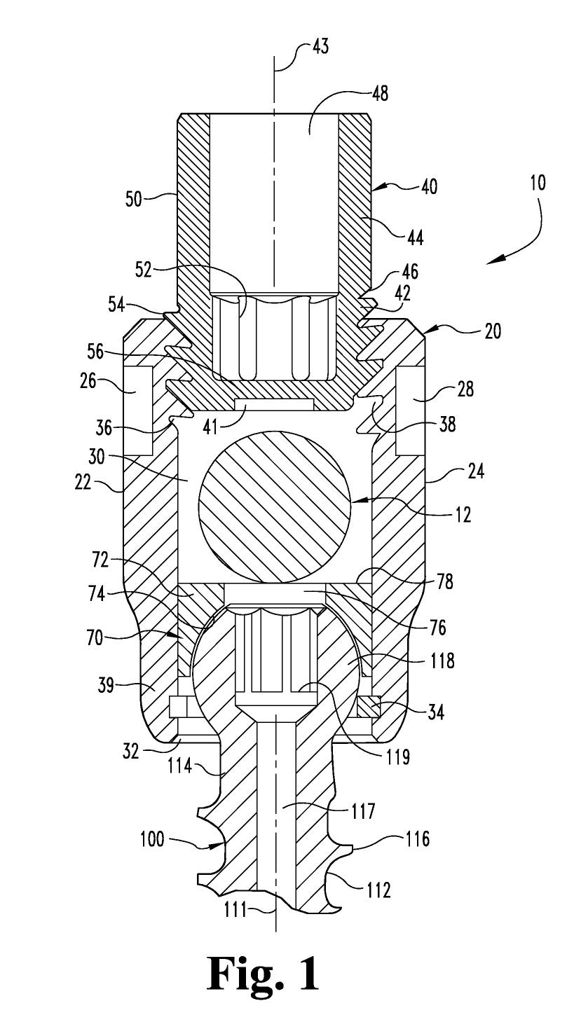

Referring to FIG. 1, there is shown a sectional view of a portion of one embodiment of an anchor assembly 10. Anchor assembly 10 includes an elongated connecting element 12 positioned through a passage 30 of a receiver 20. Receiver 20 is joined to a bone engaging portion 100 extending distally therefrom. Engaging member 40 is engaged to receiver 20 and movable therealong to contact connecting element 12 to secure it in receiver 20 with bone engaging portion 100 engaged to an underlying bony structure. Anchor assembly 10 and connecting element 12 may comprise one or more portions of a posterior spinal implant system located along a spinal column of a patient. The system may include several bone anchor assemblies 10 with at least one elongated connecting element 12 structured to selectively interconnect two or more bone anchor assemblies. Connecting elements 12 may be a spinal rod, plate, bar, or other elongated element having a length to extend between at least two vertebrae. The spinal implant systems may be used for, but are not limited to, treatment of degenerative spondylolisthesis, fracture, dislocation, scoliosis, kyphosis, spinal tumor, and/or a failed previous fusion. The spinal implant system can be affixed to posterior elements, such as the pedicles of vertebra, from a posterior approach and can be engaged to vertebrae of one or more levels of the sacral, lumbar, thoracic and/or cervical regions of the spinal column. Other embodiments contemplate that the spinal implant system is engaged along other portions of the spine, such as the anterior, lateral or oblique portions of the vertebrae V. Still other embodiments contemplate applications in surgical procedures other than spinal stabilization procedures.

As shown in FIG. 1, bone engaging portion 100 of the bone anchor can be a bone screw having proximal head portion 118 and an elongated shaft 112 (only a proximal portion shown in FIG. 1) extending distally from head portion 118 along a longitudinal axis 111. Shaft 112 can include an external thread profile 116, and can also include a lumen 117 extending axially therealong as shown. Shaft 112 can also be solid without a lumen. Lumen 117, if provided, opens into a tool recess 119 opening proximally in head portion 118 to receive a driving tool to facilitate driving bone engaging portion 100 into a bony structure, such as a vertebral body. Head portion 118 can be pivotally captured in receiver 20 with retaining member 34. The pivotal arrangement allows the multi-axial bone engaging portion to be positioned relative to the receiver and connecting element at various angles relative to one another. Such variable positioning can facilitate placement of the connecting element into receiver 20 even when the anatomical conditions prohibit or make difficult a linear arrangement of the connecting element between anchor assemblies. The engaging members 40, 40′ (FIG. 3) can enhance engagement of the connecting element 12 even if connecting element 12 is contoured through the receiver 20 to accommodate the patient's anatomy.

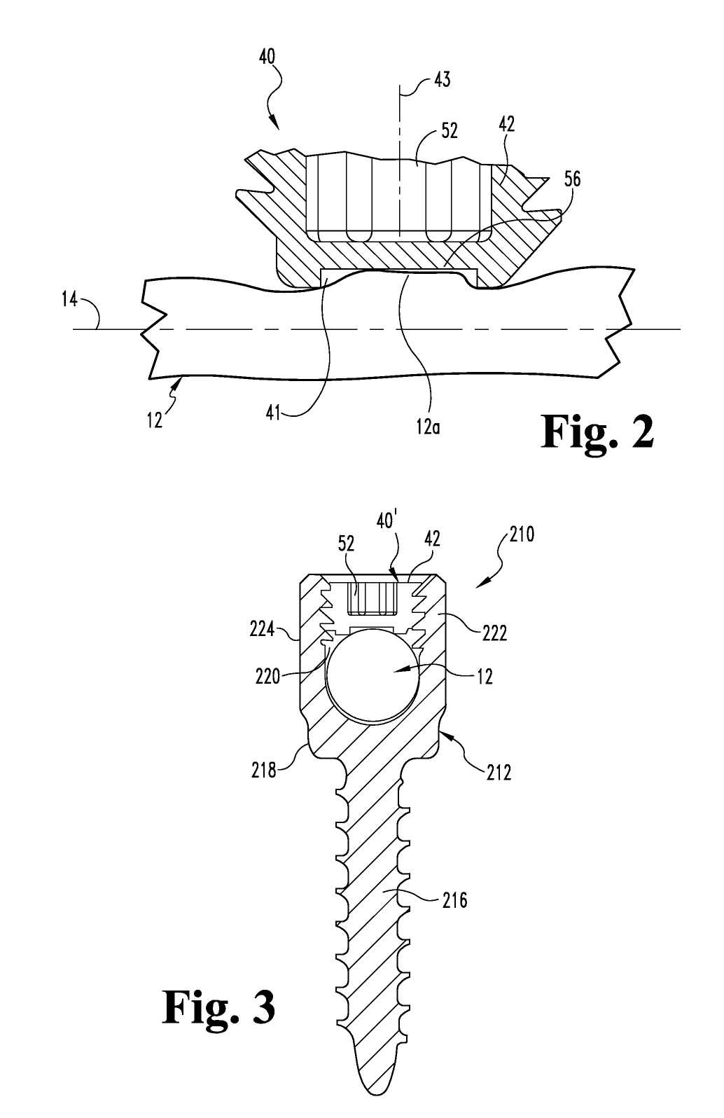

Referring further to FIG. 1, the illustrated bone anchor includes receiver 20 with a pair of arms 22, 24 extending generally parallel to one another along a central longitudinal axis 43 and defining passage 30 therebetween. Arms 22, 24 can include internal thread profiles 36, 38, respectively, to threadingly engage with engaging member 40, 40′. As shown in FIG. 2, engaging member 40 is threadingly advanced along receiver 20 to contact connecting element 12 so that a portion of connecting element 12 is deformed into cavity 41. Arms 22, 24 can also include external recesses 26, 28, respectively, to provide locations for a tool to grasp receiver 20 and/or anchoring assembly 10 during surgery. Arms 22, 24 extend proximally from a lower bowl portion 39 which pivotally houses proximal head portion 118 of bone engaging portion 100. Bowl portion 39 defines a distal opening 32 in communication with passage 30, and bone engaging portion 100 extends through distal opening 32. A retaining member 34 can axially retain bone engaging portion 100 in receiver 20 without letting head portion 118 pass through distal opening 32. Retaining member 34 can be a separate component from receiver 20, such as a split ring or C-ring, or can be formed as an integral portion of receiver 20, such as a flange or a lip about distal opening 32.

Bone anchor assembly 10 may also include a seat member 70 that can be provided adjacent to head portion 118 of bone engaging portion 100 between connecting element 12 and head portion 118. Seat member 70 includes a body 72 having a concavely curved lower surface 74 for receiving head portion 118, and an opposite seating surface 78 against which connecting element 12 can be secured. Seat member 70 can also include a central aperture 76 through which a driving tool can be positioned to engage head portion 118 and secure anchor member 100 to the underlying bony structure. Seating surface 78 can be flat, as shown, or can include a concave curvature or other shape to at least partially receive connecting element 12 thereagainst.

One embodiment a bone anchor that is multi-axial in form has been described with respect to FIG. 1. However, other embodiments contemplate other types of bone anchors. For example, FIG. 3 shows a bone anchor 210 in which receiver 218 is fixed or uni-axial relative to bone engaging portion 216. The uni-axial anchor assembly provides a fixed positioning of the receiver relative to the bone engaging portion. In FIG. 3, bone anchor 210 includes a bone engaging portion with an externally threaded shaft 216 integrally formed or structured with receiver 218 as a unit. Receiver 218 includes arms 222, 224 that form passage 220 to receive connecting element 12 therethrough. Connecting element 12 can seat against the bottom of receiver 218 in passage 220 by application of force with engaging member 40′. Arms 222, 224 can be internally threaded to threadingly engage engaging member 40, 40′ in engagement with connecting element 12. Engaging member 40′ is identical to engaging member 40, but has its proximal break-off portion removed, as discussed further below, or is provided without a proximal break-off portion.

Other embodiments contemplate other forms for the bone engaging portion of the anchor member. For example, the distal bone engaging portion can be in the form of a hook, staple, cable, tether, suture anchor, interbody fusion implant, artificial disc implant, bolt, or other structure engageable to bony tissue. The receiver defines a passage that receives a connecting element, such as a rod, tether, wire, cable, plate or other elongated linking member that can extend between one or more additional anchor assemblies secured to one or more additional vertebrae or other bony structure. The bone engaging portions can be configured as pedicle screws, bolts or other member sized and configured for engaging a pedicle of vertebra. The bone engaging portions can also be configured to engage other parts of a vertebra, or other bony structures in the patient. Furthermore, a set screw, washer, crown, cap or other device may be provided in addition to engaging member 40, 40′ for engagement within and/or about receiver to secure connecting element 12 thereto.

Receiver 20, 218 can be configured to receive connecting element 12 in passage 30, 220 with connecting element 12 top-loaded into receiver 20, 218 or loaded into receiver 20, 218 in an end-wise manner. Alternatively, the receiver can be configured so that the connecting element can be side-loaded or bottom loaded therein prior to engagement with engaging member 40, 40′. Furthermore, connecting element 12 can be engaged to two or more anchor assemblies 10, 210 along the spinal column, and provide stabilization for multiple spinal motion segments. Connecting element 12 can be a spinal rod connectable to one or more anchor assemblies to rigidly stabilize the spinal column. Connecting element 12 can also be flexible to allow motion of the spinal motion segment or segments to which it is attached. It is also contemplated that connecting element 12 can comprise multiple components. Various forms for the connecting element 12 are contemplated, including plates, wires, struts, cables, and other devices capable of engagement in a receiver of an anchor assembly with engaging members 40, 40′. Connecting element 12 can be a spinal rod comprised of any one or combination of metal, metal allow, plastic, polymer, tissue, fabric, or mesh material, for example.

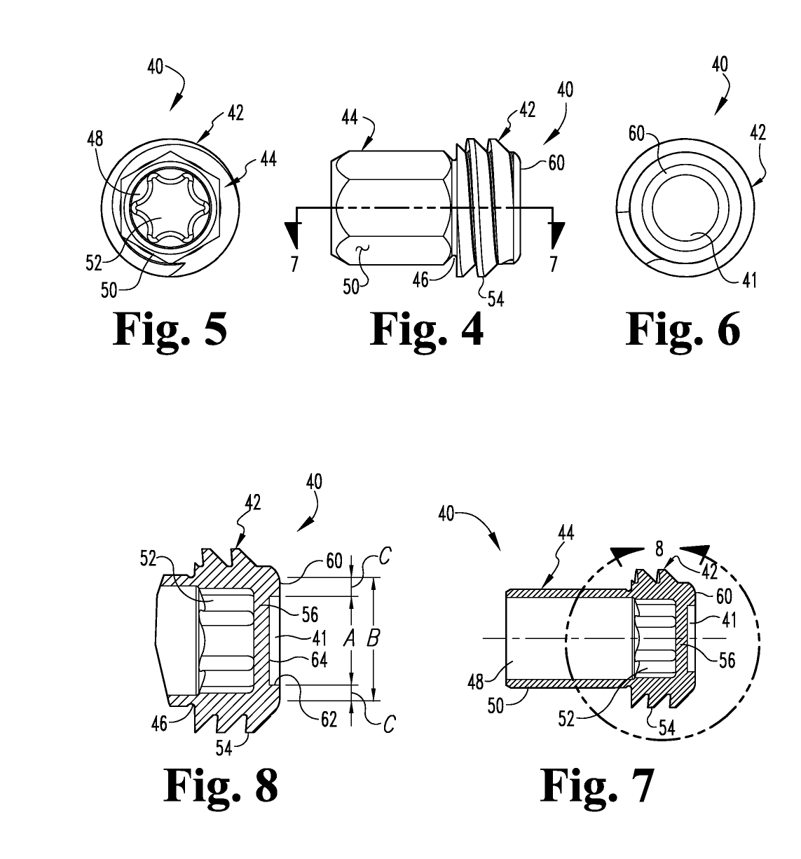

Referring further to FIGS. 4-8, engaging member 40 includes a base portion 42 for engaging receiver 20 and a proximal portion 44 extending proximally from base portion 42. Embodiments without proximal portion 44 are also contemplated, and could be configured such as shown with engaging member 40′ in FIG. 3. In the illustrated embodiment, base portion 42 includes a cylindrical body with an external thread profile 54 configured to threadingly engage the receiver, such as thread profiles 36, 38 along arms 22, 24. Proximal portion 44 is configured like a cylinder with an outer tool engaging profile 50 and a smooth inner bore 48 extending axially therethrough along a central rotation axis 43 of engaging member 40. As shown in FIG. 5, profile 50 provides an external hexagon shape to receive a driving tool (not shown) thereover and facilitate application of rotational forces to engage engaging member 40 firmly against connecting element 12. A break-off region 46 between base and proximal portions 42, 44 can be provided to allow proximal portion 44 to be removed upon application of a torque exceeding the threshold torque, leaving only base portion 42 engaged to the receiver, such as shown with respect to engaging member 40′ in FIG. 3.

First portion 42 can also include an inner tool recess 52 extending axially therein distally from bore 48 and about rotational center 43. Tool recess 52 can be engaged by a tool to allow further tightening or removal of base portion 42 when engaged to arms 22, 24 even if proximal portion 44 is removed or is not provided. Tool recess 52 can be smaller in cross-sectional size than bore 48 so that a tool sized to be received only in bore 48 cannot pass into distal tool recess 52. In the illustrated embodiment, tool recess 52 includes a hexagonal-type shape with rounded lobes to receive a driving tool, although any suitable driving recess is contemplated. Break-off region 46 is located slightly proximally of tool recess 52 so that base portion 42 remains intact when sufficient torque is applied to remove proximal portion 44. The threshold torque can be controlled or varied by controlling or varying the wall thickness at break-off region 46 during manufacture of engaging member 40. Profile 50 and recess 52 can include any suitable configuration for engagement with a driving tool to deliver driving forces to engaging member 40, including a hex shape, star shape, cross-shape, slotted shape, or other non-circular shape. Other embodiments contemplate one or more of the profile 48 and recess 52 could be omitted from engaging member 40. Still other embodiments contemplate a base portion that is internally threaded to engage the receiver, or includes non-threaded structures to engage the receiver.

First portion 42 includes a distal end that defines a pressure surface 60 positionable against connecting element 12 when connecting element 12 is located in passage 30 of receiver 20 or passage 220 of receiver 218. The distal end also defines cavity 41 extending proximally therein from pressure surface 60 to a material barrier 56 located between cavity 41 and tool recess 52. Pressure surface 60 presses against the outer surface of connecting element 12 as engaging member 40, 40′ is driven against connecting element 12. As shown in FIG. 2, connecting element 12 is compressed by the pressure surface 60 between engaging member 40, 40′ and the seating surface of the anchor member. Connecting element 12 includes a portion 12a between the opposite portions of pressure surface 60 that deforms into cavity 41. Pressure surface 60 is provided with a sufficient width orthogonally to central axis 43 to prevent the engaging member 40, 40′ from penetrating or cutting into connecting element 12. Cavity 41 is devoid of projections or material of engaging member 40 so allow the deformed portion of connecting element 12 to be fully received therein. The axial movement of connecting element 12 and/or the bone anchor along central axis 14 of connecting element 12 is resisted by the frictional forces generated by the pressure face 60 against connecting element 12 and by the axial shear force resistance provided by the material 12a of connecting element 12 that is deformed into cavity 41. The shear force resistance generated by the material of deformed portion 12a of connecting element 12 reduces the amount of torque necessary to be applied as compared to an engaging member that relies solely on frictional engagement between the engaging member and connecting element to resist axial movement along axis 14. Thus, the amount of torque that is required to be applied to engaging member 40, 40′ can be reduced while providing the same level of fixation and preserving the structural integrity of connecting element 12. The total stress introduced into the bone anchor assembly 10 by the application of torque to engaging member 40, 40′ is also reduced.

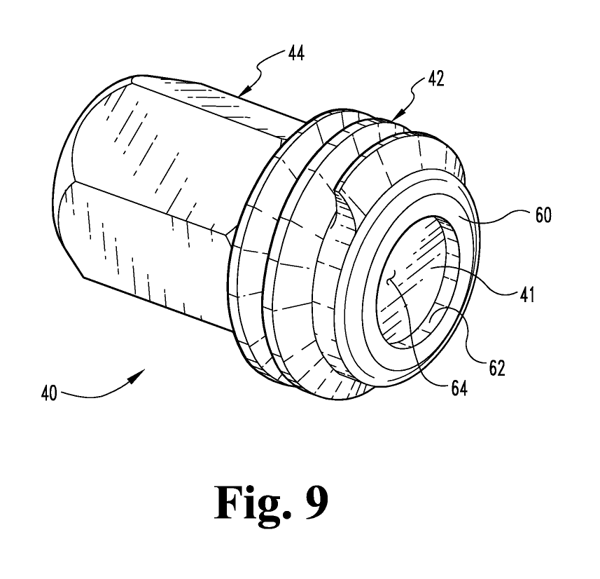

As also shown in FIG. 9, cavity 40 includes a cylindrical shape that defines a sharp cornered recess or cavity in base portion 42. Pressure surface 60 is planar and orthogonal to central rotation axis 43. Cavity 41 includes a side wall 62 that is orthogonal to pressure surface 60, and an inner or bottom surface 64 that is orthogonal to side wall 62. This configuration maximizes the volume of cavity 41 to receive deformed material from the connecting element 12. In one particular embodiment, cavity 41 includes a depth of about 0.5 millimeters. Other embodiments contemplate other depths for cavity 41 ranging from more than zero to less than that required to preserve the material barrier 56 between recess 52 and cavity 41.

Cavity 41 is centered on central rotation axis 43, and pressure surface 60 is planar and is concentric about cavity 41. In one embodiment, cavity 41 defines a width with a first dimension A orthogonally to central axis 43, and pressure surface 60 defines an overall width having a second dimension B orthogonally to central axis 43. Dimension A can range from more than zero to the major diameter of thread profile 54. In the illustrated embodiment, first dimension A is sized to provide pressure surface 60 with a connecting element contact width 2C along the distal face that corresponds to dimension B less dimension A. In one particular embodiment, the ratio of the contact width 2C of the pressure surface 60 along the distal face is about one third of width A of cavity 41 along the distal face so that the pressure exerted by pressure surface 60 is sufficient to deform the connecting element while the area and volume of the cavity 41 is sufficient to receive deformed material. Other embodiments contemplate other ratios for width 2C to width A, ranging from more than zero to one or more. In one particular embodiment, pressure surface 60 defines an area around central rotation axis 43 that is about three-fourths of the area of cavity 41. Other embodiments contemplate other ratios for the area of pressure surface 60, ranging from more than zero to one or more.

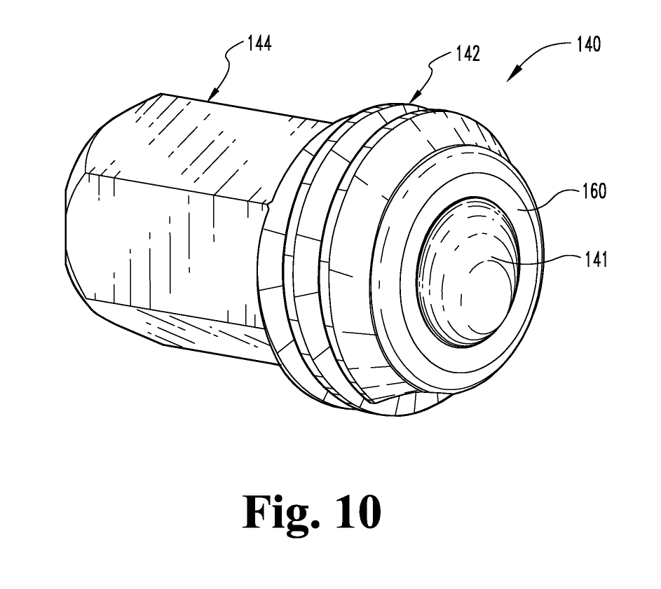

As shown in FIG. 6, cavity 41 includes a shape that is a circle when viewed in a direction looking along central axis 43. Other embodiments contemplate other shapes for cavity 61, including oval and other non-circular shapes when viewed in a direction along central axis 43. Still other embodiments contemplate the cavity 42 defines a volume that is non-cylindrical. For example, as shown in FIG. 10, engaging member 140, which can include any of the features of engaging members 40, 40′ discussed above, includes a distal portion 142 and a proximal portion 144. Distal portion 142 includes a distal end with a pressure surface 160 and a cavity 141 therein that includes a rounded or blended surface configuration. In one embodiment, cavity 141 forms a shape that defines a part of a sphere in distal portion 142. In one specific embodiment, the spherical cavity has a maximum depth of 0.5 millimeters and a curvature defined by a sphere with a 6.35 millimeter diameter. In other embodiments, pressure surface 60, 160 can be provided with grip enhancing features, such as knurlings or surface roughenings created by grit-blasting, chemical etching, machining, or other suitable technique. In still other embodiments, pressure surface 60, 160 can be non-planar.

Materials for the bone engaging portions, receivers, saddles, and engaging members disclosed herein can be chosen from any suitable biocompatible material, such as titanium, titanium alloys, cobalt-chromium, cobalt-chromium alloys, or other suitable metal or non-metal material. Connecting element 12 can be made from the same material as one or more of the components of the anchor assembly to which it is engaged, or from a different material. For example, connecting element 12 can be made from PEEK, plastic, titanium or titanium alloy, cobalt-chrome, composite material, or other material that is the same or different from the material of one or more components of the anchor assembly to which is engaged. In one embodiment, the engaging member 40, 40′ is made from metal material and the connecting element 12 is made from a polymeric material that is more readily deformed into cavity 41 by pressure applied with pressure surface 60.

Although various embodiments have been described as having particular features and/or combinations of components, other embodiments are possible having a combination of any features and/or components from any of embodiments as discussed above. As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the terms “proximal” and “distal” refer to the direction closer to and away from, respectively, an operator (e.g., surgeon, physician, nurse, technician, etc.) who would insert the medical implant and/or instruments into the patient. For example, the portion of a medical instrument first inserted inside the patient's body would be the distal portion, while the opposite portion of the medical device (e.g., the portion of the medical device closest to the operator) would be the proximal portion.

While the invention has been illustrated and described in detail in the drawings and foregoing description, the same is to be considered as illustrative and not restrictive in character, it being understood that all changes and modifications that come within the spirit of the invention are desired to be protected.

What is claimed is:

1. A spinal implant assembly, comprising:
a bone anchor member including a distal bone engaging portion engageable to a spinal column and a receiver at a proximal end of said bone engaging portion, said receiver defining a passage;
an elongate connecting element positionable along the spinal column through said passage of said receiver; and
an engaging member engageable with said receiver, wherein said engaging member extending along a central rotation axis between a distal end and a proximal end, said distal end of said engaging member including a pressure surface extending around said rotation axis, said pressure surface extending around a cavity, said cavity extending proximally from said pressure surface into said engaging member to an end wall in said engaging member that is recessed from said pressure surface, wherein said cavity receives a portion of said connecting element that deforms into said cavity when said pressure surface is compressingly engaged to said connecting element in said receiver.

2. The assembly of claim 1, wherein said pressure surface is planar and configured to avoid penetrating said connecting element when compressingly engaged against said connecting element so that said connecting element is fixed to said anchor member.

3. The assembly of claim 1, wherein said receiver includes an internal thread profile and said engaging member includes an external thread profile threadingly engageable with said internal thread profile of said receiver.

4. The assembly of claim 1, wherein engaging member includes a first portion having a cylindrical body with external threads extending thereabout and a second portion extending from said first portion, wherein said second portion is severable from said first portion upon application of a threshold torque to said second portion relative to said first portion.

5. The assembly of claim 4, wherein said first portion includes a first tool recess extending from a proximal end of said first portion along said central rotation axis, said first tool recess being configured to engage a driving tool, and said first portion includes a wall between said tool recess and said cavity that forms a material barrier between said tool recess and said cavity.

6. The assembly of claim 1, wherein said connecting element is a polymeric spinal rod.

7. The assembly of claim 6, wherein said cavity defines a cylindrical shape with a side wall extending into said engaging member orthogonally to said pressure surface and said end wall is orthogonal to said side wall.

8. The assembly of claim 1, wherein said pressure surface is planar.

9. The assembly of claim 8, wherein said cavity defines a spherical shape.

10. A spinal implant assembly, comprising:
an anchor member with a distal bone engaging portion engageable to a vertebral body and a receiver at a proximal end of said bone engaging portion, said receiver defining a passage and an thread profile extending along said passage;
an elongated connecting element in said passage of said receiver, said connecting element being structured to extend outwardly from said passage for positioning along at least two vertebrae of a spinal column; and
an engaging member threadingly engageable with said thread profile of said receiver, wherein said engaging member extends along a central rotation axis between a proximal end and a distal end, wherein said distal end includes a pressure surface extending around a cavity, wherein said cavity is located on said central rotation axis and extends from said pressure surface proximally into said engaging member, wherein said engaging member is threadingly engageable to said receiver to contact said pressure surface with an outer surface of said connecting element to compress said connecting element between said receiver and said pressure surface and deform a portion of said connecting element into said cavity without penetrating said connecting element.

11. The assembly of claim 10, wherein said cavity defines a cylinder with a side wall extending around said central rotation axis to a bottom surface of said cavity recessed proximally from said pressure surface, said bottom surface extending orthogonally to said side wall.

12. The assembly of claim 11, wherein said pressure surface is orthogonal to said central rotation axis and said side wall is orthogonal to said pressure surface.

13. The assembly of claim 10, wherein said cavity defines a cylindrical shape.

14. The assembly of claim 10, wherein said pressure surface is planar.

15. The assembly of claim 10, wherein said pressure surface defines a first width orthogonally to said central rotation axis for contacting said connecting element and said cavity defines a second width orthogonally to central rotation axis, said first width being about one third of said second width.

16. The assembly of claim 10, wherein said pressure surface defines a first area around said central rotation axis for contacting said connecting element and said cavity defines a second area located on said central rotation axis, said first area being about three-fourth of said second area.

17. The assembly of claim 10, wherein said pressure surface and said cavity occupy all of said distal end of said engaging member.

18. The assembly of claim 10, wherein said engaging member includes an externally threaded portion and a tool recess extending into said threaded portion along said central rotation axis from a proximal end of said threaded portion, said tool recess being configured to engage a driving tool, and said threaded portion includes a wall between said tool recess and said cavity that forms a material barrier between said tool recess and said cavity.

19. The assembly of claim 10, wherein said pressure surface is planar and orthogonal to said central rotation axis and said cavity defines a spherical shape.
